(19) United States
(12) Patent Application Publication
(10) Pub. No.: US 2010/0113862 A1
(43) Pub. Date: May 6, 2010

(54) TREATMENT OF AMELIORATION OF ARTHRITIC JOINT PAIN

(76) Inventors: Alan W. J. Kotowich, Delray Beach, FL (US); Todd Whitla, Winnipeg (CA)

Correspondence Address: ADE & COMPANY INC., 2157 Henderson Highway, WINNIPEG, MB, R2G1P9, CA

(21) Appl. No.: 12/265,006

(22) Filed: Nov. 5, 2008

(51) Int. Cl. A61N 2/02

(52) U.S. Cl. 600/13

(57) ABSTRACT

An apparatus for treatment of pain includes a coil formed by a generally flat elliptical base member wrapped by wire in two separated grooves spaced axially along the outer surface and a portable battery powered generator unit for generating a current such that the electromagnetic field has a required magnitude of magnetic flux density in the range 1×104⁻⁶ gauss to 1×10⁻¹² gauss at a frequency of between 0.01 and 100 Hz. The base member of the coil is covered by a flexible fabric covering material with a padding layer on each side. The coil is elliptical in plan to generate a field of the required magnitude emitted outwardly to one side in an area of the order of 10 inches by 5 inches at a depth of 2 inches. The generator is controlled wholly by simple switches which only select from a plurality of treatment options.

### FIELD OF THE INVENTION

The invention is directed to an apparatus for the treatment of chronic pain and, more particularly, to a method of ameliorating joint pain in arthritic individuals.

### BACKGROUND OF THE INVENTION

The known methods of treating chronic pain often take one—or several—approaches, including:

Prescription drugs

Over-the-counter medicines

Complementary/Alternative Medicine (CAM, also called integrative medicine)

Narcotic painkillers like OxyContin, Percocet, and Vicodin are widely associated with chronic pain relief—and with addiction.

Cox-2 inhibitor painkillers like Bextra, Celebrex, and Vioxx—pose a risk of heart attacks and strokes. (Bextra also poses a risk of serious skin reactions). Only Celebrex is still available, and the FDA requires it to carry a strong risk warning.

Surgery or implanted devices—like pain pumps—are sometimes helpful for severe chronic pain, though of course surgery has its own risks.

Over-The-Counter (OTC) drugs often begin with occasional use of a nonprescription painkiller that turns into a daily ritual of downing a handful of pills.

Some of the most problematic are NSAIDs (nonsteroidal anti-inflammatory drugs) like aspirin, Advil, Aleve, and Motrin. Taken regularly, they can result in serious gastrointestinal bleeding and may also raise the risk of cardiovascular problems. And, the older you are, the more likely you are to have complications from these drugs.

Unconventional or Complementary/Alternative Medicine therapies (CAM) are therapies not usually provided by physicians or other conventionally trained health care providers. They may be used along with conventional therapies (complementary) or instead of conventional therapies (alternative). CAM includes options like:

Acupuncture

Massage

Biofeedback

Energy therapy

Focused relaxation

Meditation

Exercise, and other techniques

Some approaches, like acupuncture, biofeedback, and massage have been shown to temporarily help ease pain in scientific studies. Other approaches have no supporting evidence, including supplements—like vitamins, herbs, and botanicals—that can have serious side effects and interact with other drugs.

The known methods of treating chronic pain are plagued with harmful contraindications, or, when sufficient data is available, have been shown to provide temporary relief at best.

Therefore, there is a need for a method of treating chronic pain to provide long-term efficacy, safely and non-invasively. There has further been a need for a method that is easy to use, and can be performed by pain sufferers without the assistance of others.

PicoTesla electromagnetic fields are produced by biologically realistic neuronal currents of 1 microampere (μA). They have been measured approximately 10 mm from their neuronal source with Superconducting Quantum Interference Devices (SQUID) since the early 1970s (Cohen). Anninos, Atienza, Goodwin, Jacobson, Sandyk, Saxena and others have since demonstrated that external exposure to very weak, non-ionizing electromagnetic fields will regenerate nerve cells; control the proliferative rate, directional attitude, and molecular genetic expression of normal human neural cells; ameliorate neurological disorders including epilepsy, Parkinson's disease, depression, MS and migraine headache; ameliorate pain. Singer demonstrated that innervation is required for a regenerative response, and Muneoka et al demonstrated that when they deviated a nerve to a wound site fibroblasts were induced to form regenerative blastema.

U.S. Pat. No. 6,099,459 (Jacobson) issued Aug. 8, 2000 discloses a magnetic field generating device and method of generating and applying a magnetic field for treatment of specified conditions. This states in the text that: “If the patient is still suffering pain, an additional signal of 0.274 volts at 7.7 Hz is added for an additional ten (10) minutes” and that “Inflammatory, including osteoarthritis, was treated. Treatment was conducted by applying a field with the system of FIG. 1 for a total of 42 minutes in the following varying amplitudes and frequencies in six minute sub periods.” This also discloses treating pain and arthritis.

US Application No: 2007/0276440A1 (Jacobson) published Nov. 29, 2007 discloses a method of treatment or prevention of cardiac diseases and conditions where an electromagnetic field is generated having a magnetic flux density from about 5×10⁻⁶ gauss to about 1×10⁻¹² gauss and a frequency of between zero and about 140 Hertz, wherein the electromagnetic field is applied therapeutically.

U.S. Pat. No. 5,453,072 (Anninos) issued Sep. 26, 2005 discloses a magnetic field generating device for treatment of epilepsy.

The disclosures of the above patents and application are incorporated herein by reference.

### SUMMARY OF THE INVENTION

It is one object of the invention to provide an apparatus for treatment of pain which is simple, inexpensive and easy to use allowing it to be used in the home.

According to a first aspect of the invention there is provided an apparatus for use in treatment or prophylaxis of pain in a human patient comprising;

a coil for generating from a current in the coil an electromagnetic field emitted by the coil for application of the field to a body part of the patient;

a generator unit for generating a current for application to the coil to generate the electromagnetic field;

the generator unit and the coil being arranged such that the electromagnetic field having a magnetic flux density providing a required magnitude in the range from 1×10⁻⁶ gauss to 1×10⁻¹² gauss and a frequency of between 0.01 and 100 Hz and arranged for application therapeutically to the body part; and

wherein the coil comprises a base member defining a top surface and a bottom surface generally parallel to the top surface;

wherein the coil is arranged such that the field at the required magnitude projects outwardly from the top surface;

and wherein the coil is covered by a flexible covering material;

and wherein there is provided a padding layer on at least the top surface of the base member allowing the coil to rest against the body part of the patient with the covering material separating the coil from the body part and the padding providing comfort for the body part.

Preferably there is provided padding on both the top and bottom surfaces allowing the device to be inverted for use on either side.

Preferably the padding is shaped at ends of the top surface so as to be of increased thickness to provide a curved surface of the covering material on the top surface of the coil. The bottom can then be flat providing different surfaces on each side for different contact conditions.

Preferably the base member includes a plastic cylindrical rim with bridging members connecting across the cylindrical rim. The cylindrical rim may be circular in plan to form a circular cylinder but is more preferably elliptical.

Preferably the base member includes a planar stiff cover sheet extending across the top face of the rim with the padding over the cover sheet.

Preferably the covering material covers both the top and bottom surfaces and optionally around the edges to provide the appearance and effect of a cushion.

Preferably the coil is arranged such that the field at the required magnitude projects outwardly from the top face of base member by 2-3 inches.

Preferably the coil is arranged such that the field at the required magnitude projects outwardly from the top face, and generally also symmetrically from the bottom face, to a distance of at least 2 inches over an area having a transverse dimension of at least 5 inches.

Preferably the area has a transverse dimension in a direction at right angles to the first transverse direction which is greater which may be of the order of 11 inches which has been found to provide an active area which conveniently covers areas to be treated.

Preferably the base member comprises a cylindrical peripheral wall with two separate grooves spaced axially along the wall each receiving a plurality of turns of wire.

Preferably the grooves are arranged adjacent top and bottom faces of the peripheral wall.

Preferably the turns of wire of one groove are connected in series with those of the other groove.

Preferably the base member is elliptical in plan view.

Preferably the generator unit is portable and battery powered.

Preferably the generator unit carries a plurality of manually operable switch positions and wherein the generator unit is arranged to generate a plurality of different treatment protocols each associated with a respective switch position for selection thereby.

Preferably the switch positions are defined by a plurality of separate touch switches so that more preferably for simple construction and operation the manually operable control switches of the device consist solely of the treatment selection switches.

According to a second aspect of the invention there is provided an apparatus for use in treatment or prophylaxis of pain in a human patient comprising;

a coil for generating from a current in the coil an electromagnetic field emitted by the coil for application of the field to a body part of the patient;

a generator unit for generating a current for application to the coil to generate the electromagnetic field;

the generator unit and the coil being arranged such that the electromagnetic field having a magnetic flux density providing a required magnitude in the range from 1×10⁻⁶ gauss to 1×10⁻¹² gauss and a frequency of between 0.01 and 100 Hz and arranged for application therapeutically to the body part; and

wherein the coil comprises a base member defining a top surface and a bottom surface generally parallel to the top surface;

wherein the coil is arranged such that the field at the required magnitude projects outwardly from the top surface;

and wherein the base member comprises a cylindrical peripheral wall with two separate grooves spaced axially along the wall each receiving a plurality of turns of wire.

According to a third aspect of the invention there is provided an apparatus for use in treatment or prophylaxis of pain in a human patient comprising;

a coil for generating from a current in the coil an electromagnetic field emitted by the coil for application of the field to a body part of the patient;

a generator unit for generating a current for application to the coil to generate the electromagnetic field;

the generator unit and the coil being arranged such that the electromagnetic field having a magnetic flux density providing a required magnitude in the range from 1×10⁻⁶ gauss to 1×10⁻¹² gauss and a frequency of between 0.01 and 100 Hz and arranged for application therapeutically to the body part; and

wherein the coil comprises a base member defining a top surface and a bottom surface generally parallel to the top surface;

wherein the coil is arranged such that the field at the required magnitude projects outwardly from the top surface;

wherein the generator unit is portable and battery powered.

The apparatus as described hereinafter is arranged therefore for restoring the electromagnetic profile of diseased and/or injured tissues through external exposure to picotesla or nanoGauss electromagnetic fields, which emulate the intensity and frequency of the physiologic fields produced by neuronal impulses. This method is applied to restore normal signal transduction of neurons, and to promote neuronal cell regeneration. The body's electromagnetic profile and its chemistry are intimately related; all biochemical events require electromagnetic interactions that promote physiologic balance. Neuronal electromagnetic fields are integral to the biological homeostasis and symmetry required to mitigate disease and disorder. This apparatus generates specifically targeted physiologic electromagnetic fields that have been shown to ameliorate chronic pain. It is portable, ergonomically designed for comfort and provides automatic, easy to use, therapy treatments. With this invention the discovery and research of physiologic electromagnetic fields moves from speculative science to practical application.

The device thus acts by externally applying supplemental physiologic electromagnetic energy to diseased or disordered tissues for the amelioration of chronic pain and for the restoration of homeostatic bio-electromagnetic and bio-chemical processes. The intensity and frequency of electromagnetic radiation can be selected using the information in the above patents and papers in the art as is well known to a person skilled in this art, emulating the radiation generated by neuronal impulses, with a specifically designed device, to provide therapeutic relief. The device is safe, effective and easy to use by patients as and when needed,

In view of the above described disadvantages of the known methods of treating chronic pain, the objectives which may be met by the device described hereinafter include;

the non-invasive mitigation of chronic pain,

a completely safe modality producing no contra-indications,

a durability of action that achieves sustained, long-term relief,

an easy to use device that is ergonomic, comfortable and portable.

To achieve the objectives of the invention very low amplitudes and frequencies are generated and applied to a coil antenna that emits electromagnetic fields. The coil is placed on the affected body tissues to externally expose them to the emitted fields.

As far as the design of the apparatus described herein is concerned, the coil configuration is unique in that it projects the fields out the sides of the coils and incorporates a cushion, making the device easy to use, more comfortable and most importantly, economically viable and marketable. The device is not restricted to using a Helmholtz coil configuration that needs to surround the area being treated.

In addition, the control interface and operating instructions of the device are very easy for the average person to use, greatly increasing functionality, allowing us to provide pain relief and enhanced quality of life to a far greater number of pain sufferers. We know of no other group who has a similar portable device that people can use as needed at home.

### BRIEF DESCRIPTION OF THE DRAWINGS

One embodiment of the invention will now be described in conjunction S with the accompanying drawings in which:

FIG. 1 is a top plan view of an electronic device to perform a method of treating chronic pain in accordance with the objectives of the invention, the coil being shown with the coverings removed to show the construction of the coil itself.

FIG. 2 is a side elevational view of the device of FIG. 1.

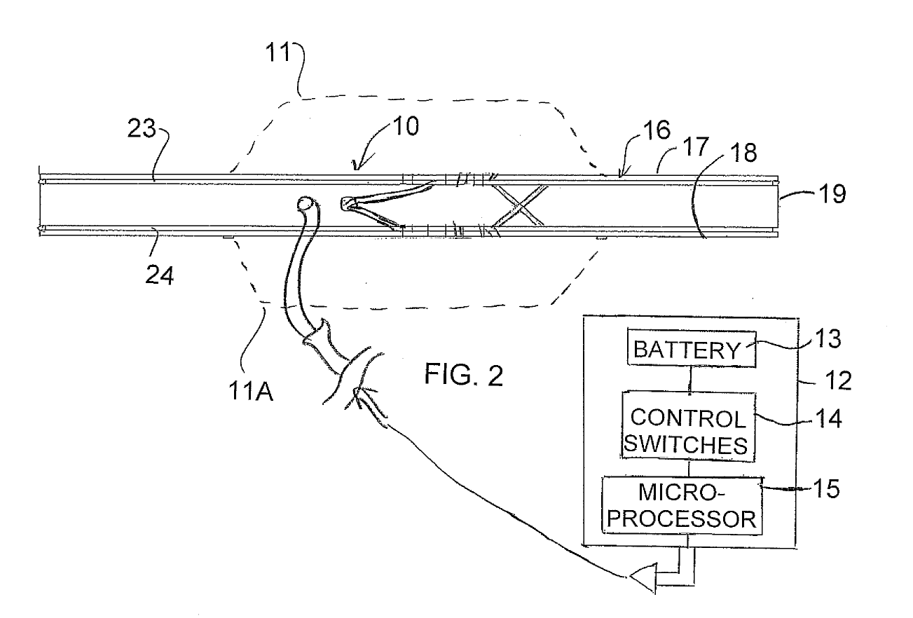

FIG. 3 is a cross sectional view along the lines 3-3 of the device of FIG. 1 with the coverings in place.

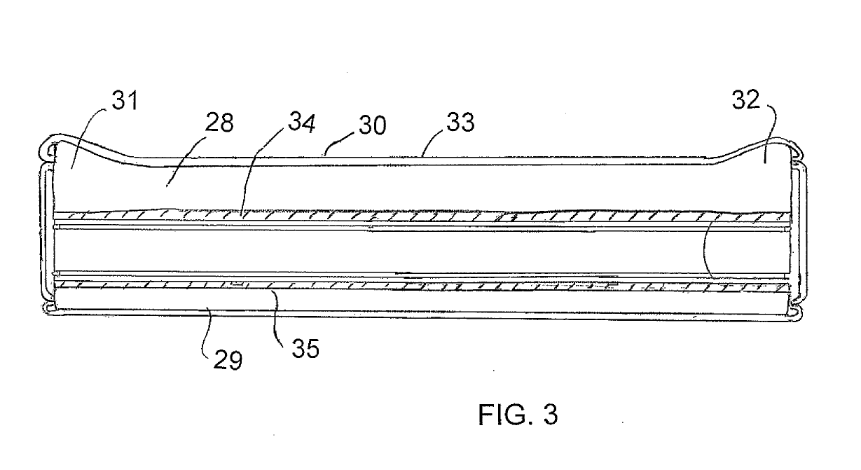

In the drawings like characters of reference indicate corresponding parts in the different figures.

### DETAILED DESCRIPTION

The apparatus shown and described herein is used for treatment or prophylaxis of pain in a human patient and includes a coil 10 for generating from a current in the coil an electromagnetic field 11, 11A emitted by the coil for application of the field to a body part of the patient.

A generator unit 12 is provided for generating a current for application to the coil to generate the electromagnetic field and includes a battery 13, manually operable switches 14 and an output current arrangement generally indicated at microprocessor 15 for generating the required currents and the required amplitude and frequency.

The generator unit and the coil are arranged such that the electromagnetic field generated by the coil has a magnetic flux density providing a required magnitude in the range from 1×10⁻⁶ gauss to 1×10⁻¹² gauss and a frequency of between 0.01 and 100 Hz. The field is arranged for application therapeutically to the body part. The field strength and frequency to be used can vary widely and is well known from the above patents and papers in this field so that no detailed disclosure is required herein.

The coil 10 comprises a base member 16 defining a top surface 17 and a bottom surface 18 generally parallel to the top surface and a generally cylindrical outer wall 19.

The base member 16 is elliptical in plan view and generates a field 11 in an area which has a transverse dimension 20 in a direction at right angles to a first transverse direction 21 where the dimension 20 is greater than the dimension 21 so as to generate an elliptical field in plan which has been found to be particularly suitable for application to many body parts.

The cylindrical peripheral wall has two separate grooves 23 and 24 spaced axially along the wall each adjacent the respective end face and each receiving a plurality of turns of wire. In one example the number of turns in each groove is of the order of 4 turns and the turns of wire of one groove are connected in series with those of the other groove.

This arrangement of coil generates therefore a field at the required magnitude which projects outwardly from the top and bottom surfaces by a distance of the order of 2-3 inches so as to extend into a body part on to which the coil is applied.

The base member 10 includes a plastics cylindrical rim defined by the wall 19 with bridging members 24 and 24A connecting across the cylindrical rim at right angles to hold the rim stiff while reducing weight of the base member.

There is provided a padding layer 28 of foam on the top surface of the base member and a similar padding layer 29 on the bottom surface of the base member. A fabric layer 30 engages over the padding layers and wraps around the whole structure with stitching at suitable locations to tailor the cover over the padding layers and the edges allowing the coil to rest against the body part of the patient with the covering material separating the coil from the body part and the padding providing comfort for the body part.

The padding 28 is shaped at ends 31 and 32 of the elliptical top surface of the top surface so as to be of increased thickness to provide a curved surface 33 of the covering material on the top surface of the coil.

Between the padding and the coil member is provided a planar stiff cover sheet 347 35 of card board or the like extending across the top face and bottom face respectively of the rim with the padding over the cover sheet.

The coil is arranged such that the field at the required magnitude projects outwardly from face to a distance of at least 2 inches over an area having a transverse dimension 21 of at least 5 inches.

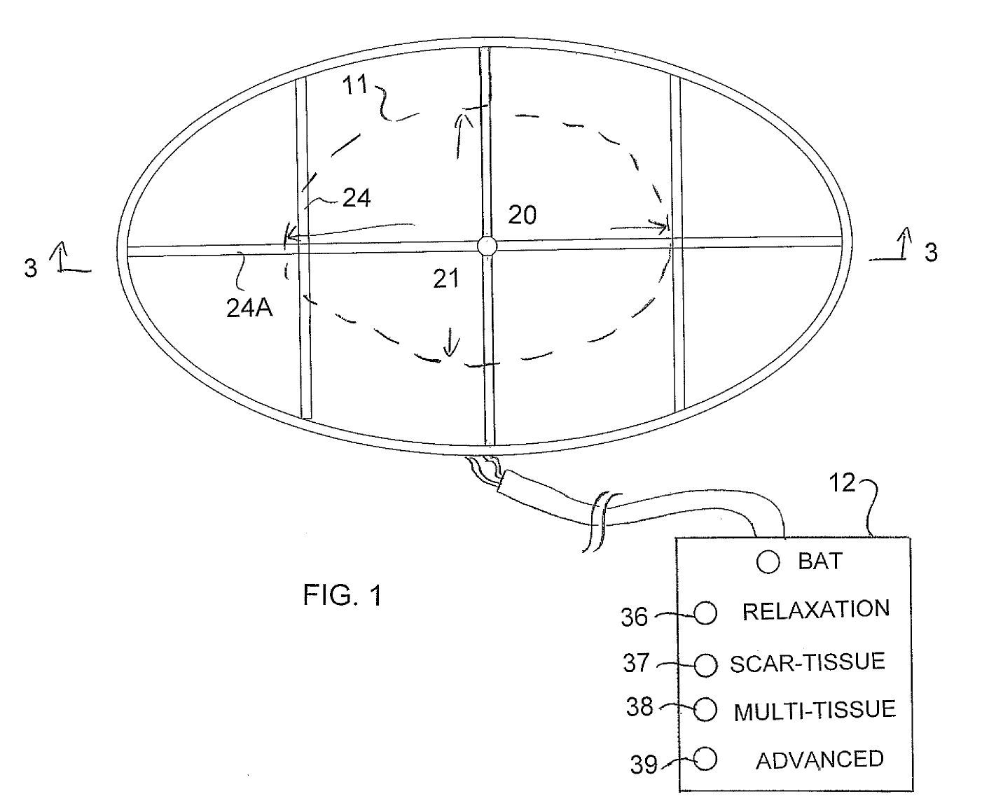

The generator unit is portable and battery powered and is operated simply by a plurality of manually operable switch positions arranged to generate a plurality of different treatment protocols each associated with a respective switch position for selection thereby. Thus as shown in FIG. 1 there are four switch positions each defined by a press switch 36, 37, 38 or 39 and each associated with a treatment such as “relaxation”, “Scar Tissue”, “Multi-Tissue” and “Advanced” for four different treatment levels. The device is turned on by selecting one of the switches and is turned off after the treatment protocol has completed as determined by the control system. Suitable variations in the amplitude and frequency of the different protocols are readily determined by a person skilled in the art from the above patents, papers and scientific dissertation. The control circuit for generating the required currents is also well apparent to any competent electrical engineer so that details of the circuit are also not required.

The generator unit is operated solely by the manually operable control switches without any variability of the protocols being available to the end user so that the device is simple and readily useable in the home by unskilled operators or by the patient.

The device produces physiologic electromagnetic fields that replace those normally emitted by healthy nerve cells. These externally applied fields reinstate orderly cellular communication and co-operation thus normalizing biochemical reactions. Damaged neurons are then regenerated resulting in renewed homeostatic neuronal impulses. Neuronal electromagnetic fields are an essential component, likely performing an executive role, in the growth and repair of living systems.

The method of treating chronic pain utilizes an electronic device as shown schematically in FIG. 1. The device includes a signal generator that produces picotesla or nanoGauss electromagnetic fields in the brain wave frequency range up to approximately 100 Hz. Switches are provided for selecting the operative field parameters for the targeted tissue. The signal generator is connected to one 14″ by 8″ elliptical coil 1.172″ wide with two winding grooves approximately 1″ apart that are wound with 4 turns of copper wire. The coil is composed of a PVC plastic and is placed within a foam cushion, which is then enclosed in a cloth cover. The coil antenna produces alternating current sinusoidal electromagnetic pulse fields that extend up to 3″ perpendicularly on both sides of the coil.

The method and the time required to perform the method, vary for individual patients, and depend on the severity of the underlying disorder that is the cause of chronic pain. In general, a greater amount of time is required when the severity is greater, and the operative field parameters vary as soft tissue, connective tissue and bone tissue are individually targeted. The method is easily performed by a patient using this device.

The effectiveness of the method of the invention is based on the selection of the correct signal parameter for the targeted tissue. The patient simply places the center of the cushioned antenna over the painful area and selects one of four prescribed treatments. The treatment choices are:

Inflammation and Relaxation (relaxation)—for the treatment of inflammation and tension.

Scar Tissue—for the treatment of early stage arthritis.

Multi-Tissue—for the treatment of arthritis that involves connective tissue.

Advanced—for the treatment of severe arthritis involving bone tissue.

Healthy tissue is unaffected because the invention exposes the body to physiologic electromagnetic fields, a feature that allows the patient to test each of the four prescribed treatments to determine which is most beneficial to their particular affliction. Treatment can therefore be performed without the need for extensive diagnostic procedures; the patient will self-diagnose their affliction based upon treatment choice response.

The signal generator is pre-programmed to provide each of the four treatment options in forty-five minute sessions. Treatments can be terminated early and recommenced as needed, however optimal results will only be achieved when a treatment session runs uninterrupted to its forty-five to fifty-five minute conclusion. Treatments can be repeated as often as necessary. Pain reduction, increased flexibility and range of motion will improve with each treatment, and generally, five to seven treatments are sufficient to produce long-term relief.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. Apparatus for use in treatment or prophylaxis of pain in a human patient comprising;
a coil for generating from a current in the coil an electromagnetic field emitted by the coil for application of the field to a body part of the patient;
a generator unit for generating a current for application to the coil to generate the electromagnetic field;
the generator unit and the coil being arranged such that the electromagnetic field having a magnetic flux density providing a required magnitude in the range from 1×10⁻⁶ gauss to 1×10⁻¹² gauss and a frequency of between 0.01 and 100 Hz and arranged for application therapeutically to the body part; and
wherein the coil comprises a base member defining a top surface and a bottom surface generally parallel to the top surface;
wherein the coil is arranged such that the field at the required magnitude projects outwardly from the top surface;
and wherein the coil is covered by a flexible covering material;
and wherein there is provided a padding layer on at least the top surface of the base member allowing the coil to rest against the body part of the patient with the covering material separating the coil from the body part and the padding providing comfort for the body part.

2. The apparatus according to claim 1 wherein there is provided padding on both the top and bottom surfaces.

3. The apparatus according to claim 1 wherein the padding is shaped at ends of the top surface so as to be of increased thickness to provide a curved surface of the covering material on the top surface of the coil.

4. The apparatus according to claim 1 wherein the base member includes a plastics cylindrical rim with bridging members connecting across the cylindrical rim.

5. The apparatus according to claim 1 wherein the base member includes a planar stiff cover sheet extending across the top face of the rim with the padding over the cover sheet.

6. The apparatus according to claim 1 wherein the covering material covers both the top and bottom surfaces.

7. The apparatus according to claim 1 wherein the coil is arranged such that the field at the required magnitude projects outwardly from the top face of base member by 2-3 inches.

8. The apparatus according to claim 1 wherein the coil is arranged such that the field at the required magnitude projects outwardly from face to a distance of at least 2 inches over an area having a transverse dimension of at least 5 inches.

9. The apparatus according to claim 8 wherein the area has a transverse dimension in a direction at right angles to the first transverse direction which is greater.

10. The apparatus according to claim 1 wherein the base member comprises a cylindrical peripheral wall with two separate grooves spaced axially along the wall each receiving a plurality of turns of wire.

11. The apparatus according to claim 10 wherein the grooves are arranged adjacent top and bottom faces of the peripheral wall.

12. The apparatus according to claim 10 wherein the turns of wire of one groove are connected in series with those of the other groove.

13. The apparatus according to claim 1 wherein the base member is elliptical in plan view.

14. The apparatus according to claim 1 wherein the generator unit is portable and battery powered.

15. The apparatus according to claim 14 wherein the generator unit carries a plurality of manually operable switch positions and wherein the generator unit is arranged to generate a plurality of different treatment protocols each associated with a respective switch position for selection thereby.

16. The apparatus according to claim 15 wherein the switch positions are defined by a plurality of separate touch switches.

17. The apparatus according to claim 15 wherein the generator unit includes manually operable control switches consisting solely of the treatment selection switches.

18. Apparatus for use in treatment or prophylaxis of pain in a human patient comprising;
a coil for generating from a current in the coil an electromagnetic field emitted by the coil for application of the field to a body part of the patient;
a generator unit for generating a current for application to the coil to generate the electromagnetic field;
the generator unit and the coil being arranged such that the electromagnetic field having a magnetic flux density providing a required magnitude in the range from 1×10⁻⁶ gauss to 1×10⁻¹² gauss and a frequency of between 0.01 and 100 Hz and arranged for application therapeutically to the body part; and
wherein the coil comprises a base member defining a top surface and a bottom surface generally parallel to the top surface;
wherein the coil is arranged such that the field at the required magnitude projects outwardly from the top surface;
and wherein the base member comprises a cylindrical peripheral wall with two separate grooves spaced axially along the wall each receiving a plurality of turns of wire.

19. Apparatus for use in treatment or prophylaxis of pain in a human patient comprising;
a coil for generating from a current in the coil an electromagnetic field emitted by the coil for application of the field to a body part of the patient;
a generator unit for generating a current for application to the coil to generate the electromagnetic field;
the generator unit and the coil being arranged such that the electromagnetic field having a magnetic flux density providing a required magnitude in the range from 1×10⁻⁶ gauss to 1×10⁻¹² gauss and a frequency of between 0.01 and 100 Hz and arranged for application therapeutically to the body part; and
wherein the coil comprises a base member defining a top surface and a bottom surface generally parallel to the top surface;
wherein the coil is arranged such that the field at the required magnitude projects outwardly from the top surface;
wherein the generator unit is portable and battery powered.

20. The apparatus according to claim 19 wherein the generator unit carries a plurality of manually operable switch positions and wherein the generator unit is arranged to generate a plurality of different treatment protocols each associated with a respective switch position for selection thereby.
